(19) United States
(12) Patent Application Publication
(10) Pub. No.: US 2007/0101455 A1
(43) Pub. Date: May 3, 2007

(54) Castanea sativa mill. genes codifying for allene oxide cyclase, cystatin, beta-1, 3-glucanase and thaumatin-like protein and their use

(76) Inventors: Susana Maria Traquete Serrazina, Lisboa (PT); Sandra Cristina Matias Fonseca, Louras (PT); Maria Salome Soares Pais, Lisboa (PT); Aladje Balde, Sintra (PT)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/562,144

(22) PCT Filed: Jun. 25, 2004
(86) PCT No.: PCT/PT04/00015

§ 371 Date: Jul. 20, 2006

(30) Foreign Application Priority Data
Jun. 26, 2003 (PT) 102979S

(51) Int. Cl. A01H 1/00; C07H 21/04; C12N 9/24; C12N 5/04; C12N 15/82

(52) U.S. Cl. 800/279; 435/419; 435/468; 435/200; 536/23.2

(57) ABSTRACT

This invention provides isolated and purified nucleotide sequences which are differentially expressed during chestnut infection with the pathogenic fungus Phytophthora cinnamomi Rands. The isolated genes can be inserted into expression cassettes and cloned in an expression vector which can be used to transform a host cell by selected transformation methods. Transgenic plants can be regenerated from transformed plant cells by in vitro culture techniques. The nucleotide sequences disclosed in this invention encode proteins which are described as having an effective action in plant resistance to pathogenic fungi. When used in sense orientation they can delay or even prevent plant infection, bringing important advantages for chestnut, cork-oak or other woody tree species' producers.

### FIELD OF THE INVENTION

The present invention relates to the isolation and identification of nucleotide sequences encoding for proteins involved in the European chestnut resistance to the pathogenic fungus Phytophthora cinnamomi, responsible for the chestnut ink disease, a method to improve the resistance by transforming plants with a construct containing one of the isolated genes, and transgenic plants and seeds transformed with such constructs.

### BACKGROUND OF THE INVENTION

European chestnut (Castanea sativa Mill.) is an important woody species with economic interest, after wood and fruit. Ecologically, the chestnut culture offers soil protection and fixation, specially in mountainous and declivous regions.

In the last decade the distribution area of European chestnut has dangerously declined due to various factors: the aging of the populations and diseases, namely the ink disease, caused by the fungi Phytophthora cinnamomi and Phytophthora cambivora, and, more recently, the cancer disease, caused by the fungus Criphonectria parasitica.

In Portugal, C. sativa suffered a serious decrease since 1886 [Malato-Beliz (1987), O castanheiro na economia e na paisagem, Edição da Câmara Municipal de Castelo de Vide], as a result of ink disease. Vieira Natividade started the selection programs to achieve tolerant clones started at the 40's, and continued in the 60's by Carvalho Fernandes [Fernandes, C. T. (1966), A “doença da tinta” dos castanheiros. Parasitas do Género Phytophthora de Bary, Dissertação de Concurso para Investigador em Patologia Vegetal, Direcção Geral dos Serviços Florestais e Aquícolas, Centro de Investigações Florestais, Alcobaça]. The majority of the clones selected on the resistance to the ink disease were lost in the last 50 years, remaining, at the present time, scarce information on the genetic identity of the survivors.

After Telhada J.A.B.M. (1990, A “tinta” do castanheiro—aspectos principais e perspectivas de lute. Vida Rural, 5, 36-39), the ink disease in adult chestnut trees is manifested by radicle and younger root darkening and by a softening of the epidermis. The roots tissues decompose, leading to a progressive decline of the branches. Dark blots are observed in the older roots and tree laps [Grente, J. (1961), La maladie de L'Encre du Chataignier I—Étiologie et Biologie. Ann. Epiphyties, 12, 5-24].

Telhada and Grente report, in the aerial zone, a leaf yellowing starting in the brunch upper parts, from above to below. The leaf blade may present slight closure in V form. The yellowish leaves do not present autumn abcission.

The P. cinnamomi infectious structures correspond to zoospores that may exist near the roots, especially in flooding conditions. The fungal penetration in the plant tissue may occur by the intercellular way, in which a germinative tube progresses between two epidermic anticlinal walls, or by the intracellular way, also by a germinative tube mode [Dale, M. L., Irwin, J. A. G. (1991), Stomata as an infection court for Phytophthora megasperma f. sp. medicaginis in chickpea and a histological study of infection, Phytopathology, 81, 375-379]. In younger roots and non-lignified stems, fungal penetration occurs through the epidermis [Whiteside, J. O. (1971), Some factors affecting the occurrence and development of root rot on citrus trees, Phytopathology, 61, 1233-1238]. In lignified, suberized, or root cap tissues, the germinative tube penetration is achieved only after wounding [Boccas, B., Laville, E. (1978), Les maladies á Phytophthora des agrumes, SETCO-IRFA Ed., Paris].

From RAPD (Random Amplified Polymorphic DNA) molecular characterization studies in tolerant clones to ink disease [Seabra, R. C., Ribeiro, G., Cotrim, H., Pais, S. (1996), First Approach for the Molecular Characterisation of Chestnut Clones, Silva Lusitana, 4(2), 251-253; Seabra, R.M.L.C. (1998), Transformação Genética de Castanea sativa Mill. e Caracterização Molecular do Género Castanea, Dissertação de Doutoramento, Faculdade de Ciências da Universidade de Lisboa], is concluded that natural populations possess a large genetic diversity. These results appoint to a scientific basis to proceed with the study of isolation and characterization of resistance genes to ink disease.

In the last years investigation has been developed with the aim to identify the genes responsible for the plant resistance to several diseases (R genes), confirming a great homology degree between them. The R genes identification and cloning may lead to their transfer to selected genotypes, by genetic transformation.

Genes codifying Allene Oxide Cyclase (AOC), Cystatin, β-1,3-Glucanase and Thaumatin-Like Protein are described of extreme importance in the defence of several plants to pathogenic micro organisms' attack.

The step catalyzed by AOC in the jasmonate biosynthetic pathway has extreme importance in wound disease in, at least, tobacco, potato and Arabidopsis. In the last decade, jasmonates have been recognised as decisive elements in a signalling cascade with lipidic basis, with a key-role in plant defence reactions against pathogens. A reference work to AOC in tobacco was published by Stenzel, I., Hause, B., Maucher, H., Pitzschke, A., Miersch, O., Ziegler, J., Ryan, C. A. e Wasternack, C. (2003), Allene oxide cyclase dependence of the wound response and vascular bundle-specific generation of jasmonates in tomato—amplification in wound signalling, The Plant Journal, 33, 577-589.

Cystatins interfere on the regulation of proteomic turnover and have an important role in the resistance against insects and pathogens. They are also designated as cystein protease inhibitors, as they inhibit proteases released by the pathogen when the plants are infected, causing great damage to host cells. In Arabidopsis leaves Cystatins are greatly induced by wounding, non-virulent pathogen attacks, or nitric oxide [Belenghi, B., Perazzolli, M., Delledonne, M. (2002), ATCYS from Arabidopsis thaliana encodes a cysteine-protease inhibitor that functions as a negative regulator of hypersensitive cell death, Proceedings of the XLVI Italian Society of Agricultural Genetics—SIGA Annual Congress, Giardini Naxos, Italy, 18/21 September, Poster Abstract 5.13]. More details about this enzyme are described in Kondo, H., Abe, K., Nishimura, I., Watanabe, H., Emori, Y. e Arai, S. (1990), Two Distinct Cystatin Species in Rice Seeds with Different Specificities against Cysteine Proteinases, The Journal of Biological Chemistry, Vol. 265, No. 26, 15832-15837.

Plant β-1,3-Glucanases are abundant proteins evolved in several physiological and developmental metabolisms, including microsporogenesis, polen germination, seed fertilization and germination and pathogen defense. Plant β-1,3-Glucanases are divided in, at least, three classes, depending on the primary structure. Class III includes glucanases induced by pathogens. The expression of many β-1,3-Glucanases may be induced by fungal elicitors, wound, salicilic acid, ethylene and other chemical inducers. β-1,3-Glucanase genes can also be expressed during the hypersensitive response in tobacco leaves inoculated with TMV virus. It is considered that β-1,3-Glucanases act directly against fungi, hydrolising wall β-1,3-glucans, or indirectly, hydrolising pathogen and host polysaccharides to produce elicitors capable to originate an hypersensitive response. β-1,3-Glucanases have been object of numberless studies, among then are the one of Cheong, Y. H., Kim, C. Y., Chun, H. J., Moon, B. C., Park, H. C., Kim, J. K., Lee, S.-H., Han, C.-D., Lee, S. Y., Cho, M. J. (2000), Molecular cloning of a soybean class III β-1,3-glucanase gene that is regulated both developmentally and in response to pathogen infection, Plant Science, 154, 71-81.

The action of several isoforms is likely evolved in the cellular membrane permeabilization of the target pathogen cell, after the specific binding of several non-water soluble Thaumatin-Like Protein units to β-1,3-glucans. Amongst other references, Thaumatin-Like Proteins are described in: Trudel, J., Grenier, J., Potvin, C., Asselin, A. (1998), Several Thaumatin-Like Proteins Bind to β-1,3-glucans, Plant Physiology, 118, 1431-1438; Darby, R. M., Firek, S., Mur, L. A. J., Draper, J. (2000), A thaumatin-like gene from Asparagus officinalis (AoPRT-L) exhibits slow activation following tissue maceration or salicylic acid treatment, suggesting convergent defence-related signalling in monocots, Molecular Plant Pathology, 1(6), 357-366.

### SUMMARY OF THE INVENTION

AOC, Cystatin, β-1,3-Glucanase e Thaumatin-Like Protein codifying genes were isolated from ink disease resistant C. sativa plants, after inoculation with the pathogenic fungus P. cinnamomi. Those genes are expressed after and during the infection and have an important role in plant defence to pathogens. The isolated genes regulate the expression of the reported enzymes and generate plants with a high degree of susceptibility to ink disease when silenced.

Those genes can be inserted in sense in chestnut or in other species of the Fagaceae (Fagus, Quercus . . . ). Therefore, economically important European chestnut varieties may achieve superior tolerance to ink disease caused by the fungus P. cinnamomi.

### DETAILED DESCRIPTION AND PREFERRED EMBODIMENTS OF THE INVENTION

The present invention provides new isolated genes from European chestnut, expressed during the infection with P. cinnamomi. These genes encode for pathogen defence signalling—AOC—, protection from fungal enzyme proteolysis—Cystatin—, fungal cell wall hydrolysis—β-1,3-Glucanase—and permeabilization of fungal cellular membrane—Thaumatin-Like Protein.

Also provided in this invention, the claimed nucleic acid sequences can be used to improve the endogenous expression of AOCCs, CystCs, GlucCs and TLPCs genes in any plant organ, increasing the tolerance to ink disease. The over expression may be achieved through “sense up regulation”. mRNA, RNA, cDNA and DNA molecules inserted in sense orientation can serve this purpose.

Nucleic Acid Sequences Isolation from Plants

The genes of the present invention may be isolated from plant inoculated leaves using different methods well known in the art. In particular, one approach can be used, the one described here. It consists on specific primer design from conserved portions of the gene of interest, isolated from the same species, published in the database. This was the case for Cystatin and Thaumatin-Like Protein genes. In the same approach, degenerated primers were designed from conserved portions of sequence alignments, using sequences from the same gene isolated from other species published in the database. This was the case for AOC and β-1,3-Glucanase genes.

The procedures for isolating the RNA or cDNA encoding a protein according to the present invention, subjecting it to isolation of the cDNA fragments, ligation of the fragments to a cloning vector and transforming a host are well known in recombinant DNA technology. Accordingly, one of ordinary skill in the art can use or adapt the detailed protocols for such procedures as found in Sambrook et. al. (1989), Molecular Cloning: A Laboratory Manual, 2nd Edition, Cold Spring Harbor, N.Y., or any other manual on recombinant DNA technology. Fragments of the genes of the present invention are also contemplated by the present invention.

The designed specific primers can be substituted by other primers aiming the isolation of slightly different cDNA fragments of the same sequences claimed here, advancing in the knowledge of the sequence. The designed degenerated primers can be used to obtain isoenzymes of the same gene in Castanea species or to isolate the homologous gene from other different species by PCR and other in vitro amplification methods. For a general overview of PCR see PCR Protocols: A Guide to Methods and Applications (Innis, M., Gelfand, D., Sninsky, J. e White, T. eds.), Academic Press, San Diego (1990).

Polynucleotide can also be synthesised by well-known techniques as described in the technical literature. Double stranded DNA fragments may then be obtained either by synthesizing the complementary strand and annealing the strands together under appropriate conditions, or by adding the complementary strand using DNA polymerase with an appropriate primer sequence.

One once coding gene of the present invention has been isolated from species, it can serve as a hybridization probe to isolate corresponding genes from other species by cross-hybridization under low or moderate stringency conditions. Used as heterologous probes, the isolated genes can be used for screening a cDNA library or a genomic library, from any species. Used as homologous probes, the isolated nucleic acid sequences can be used to screen a library constructed from any species of Castanea genus.

Use of Nucleic Acids of the Invention to Inhibit Gene Expression

According to the present invention, a DNA molecule may also be operable linked to a promoter capable of regulating the expression of the said DNA molecule, to form a chimerical gene. This chimerical gene can be introduced into a replicable expression vector, for using in transforming plants. The replicable expression vectors may also be used to obtain the polypeptides coded by the genes of the present invention by well-known methods in recombinant DNA technology.

Replicable expression vectors usually comprise a promoter (at least), a transcription enhancer fragment, a termination signal, or a combination of two or more of these elements operable linked in proper reading frame. Preferably the vector encodes also a selectable marker, for example, antibiotic resistance. Replicable expression vectors can be plasmids, cosmids, bacteriophages and viruses.

The isolated sequences can be used to prepare expression cassettes useful in a number of techniques. For example, these expression cassettes can be used to enhance the expression of endogenous AOCCs, CystCs, GlucCs and TLPCs genes. Over expression can be useful, for instance, to improve the ink disease resistance in susceptible European chestnut varieties, to signalize for defence responses (AOCCs gene), originating damage to pathogenic fungus (GlucCs and TLPCs genes), or acting on hazard effects of the pathogen (Cyst gene).

To increase gene expression in plants using sense technology, the codifying nucleic acid sequence or open reading frame can be operate linked to a promoter (CaMV35S promoter or to a root specific promoter, for example) such that the sense strand of RNA will be transcript. This expression cassette can be used to plant genetic transformation, where sense RNA strands will be transcript. A higher accumulation of mRNA codifying the interest enzyme, added to the endogenous production, will imply a higher synthesis of enzymes related to ink disease defence in susceptible varieties. On other hand, CaMV35S promoter is highly active in a wide variety of plant types, being able to supply a constitutive expression of the genes of interest, allowing an improved protection against P. cinnamomi.

Use of Nucleic Acids of the Invention to Produce Transgenic Plants

The nucleic acid sequences isolated in the present invention can be incorporated in an expression vector and thereby be introduced into a host cell. Accordingly, one skilled in the art can use the sequences to make a recombinant cell. Suitable host cells include, but are not limited to, bacteria, virus, yeast, mammalian cells, insect, plant, and the like. Preferably the host cells are either a bacterial cell or a plant cell.

The nucleotide sequences claimed in this invention can be inserted in an expression vector, which may be introduced into the genome of the desired plant host by a variety of conventional techniques. The constructions using the isolated genes can be introduced into a conventional Agrobacterium tumefaciens host vector. The virulence functions of the Agrobacterium host will direct the insertion of the construct and adjacent marker into the plant cell DNA when the bacteria infect the cell.

Alternatively, the DNA constructs can be directly introduced into the plant cell genomic DNA using techniques such as electroporation and microinjection in plant cell protoplasts. Ballistics methods, such as DNA particle bombardment, allows the DNA to be introduced directly in plant tissue.

Transformed plant cells derived by any of the above transformation techniques can be cultured to generate a whole plant, which possesses the transformed genotype and thus the desired phenotype such as increased fruit firmness. Such regeneration techniques rely on the manipulation of certain nutrients and phytohormones in a culture medium containing an antibiotic, herbicide or other marker that has been introduced together with the nucleotide sequences of interest. Regeneration can also be obtained from different plant explants or embryos. For a general overview, see Plant Cell, Tissue and Organ Culture, Fundamental Methods (O. L. Gamborg e G. C. Philips eds.), Springer-Verlag, 1995. Plant tissues suitable for transformation include, but are not limited to, floral buds, leaf tissue, root tissue, meristems, zygotic and somatic embryos, anthers, microspores and megaspores.

The introduction of genes in C. sativa genomic DNA, by Agrobacterium genomic transformation has been achieved [Seabra, R.M.L.C. (1998), Transformação Genética de Castanea sativa Mill. e Caracterização Molecular do Género Castanea, Dissertação de Doutoramento, Faculdade de Ciências da Universidade de Lisboa], although the chestnut recalcitrance to explant regeneration. Other systems with great potential were not yet tested as an alternative to chestnut transformation, as the somatic embryo particle bombardment [Seabra, R. C., Pais, M. S. (2003) Genetic Transformation of Chestnut, em Plant Genetic Engineering, Vol. 3, Singh, R. P., Jaiwal, P. K. eds., SCI Tech Publishing LLC, USA]. The genetic transformation of Quercus suber somatic embryos by particle bombardment was already achieved by Neto, H. (1995), Estudo das Condições de Cultura in vitro e de Transferência de Genes em Quercus suber L., Disseretação de Mestrado, Faculdade de Ciências da Universidade de Lisboa. Somatic embryo use in genetic transformation is profitable, as embryos are genetically unvaried organs that reproduce the characteristics of the mother plant. Regeneration by somatic embryogenesis allows surpassing many of the disadvantages of the regeneration by organogenesis (with multicellular origin) [Dunstan, D. I., Thorpe, T. A. (1986). Regeneration in Forest Trees. Cell Culture and Somatic Cell Genetics of Plant, Vol. 3].

Pathogen attack control can be achieved in the transformed plants with constructions containing the isolated cDNA sequences. The resulting transformed plants with the genes of this invention may have an over expression of AOCCs, CystCs, GlucCs or TLPCs genes. These plants may have an enhanced resistance against pathogen fungal attack, preventing, delaying or reducing the wound/damage extension.

The DNA molecules of the present invention may be used to transform any plant in which expression of the particular protein encoded by said DNA molecules is desired. The DNA molecules of the present invention can be used over a broad range of plants, but they are extremely useful to genera Castanea e Quercus.

Any skilled person will recognize that an enzymatic activity assay, immunoassay, western blotting, and other detection assays can be used to detect, at the protein level, the presence or absence of the proteins which the isolated sequences encode for. At DNA level, southern blotting, northern blotting and PCR analysis can be performed in order to determine the effective integration of the desired gene sequences in the plant DNA, and the efficient gene (endogenous and exogenous) expression due to introduced sequences.

Any skilled person will recognize that after an expression cassette being stably incorporated in transgenic plants and confirmed to be operable, it can be introduced into other plants by sexual crossing. A number of standard breeding techniques can be used, depending on the species to be crossed. Transgenic seeds and propagules (e.g., cuttings) can be obtained and, when cultured, produce transgenic plants.

The embodiments described above and the following examples are provided to better illustrate the practice of the present invention, and should not be used to limit the scope of the invention. It is understood that the invention is not restricted to the particular material, combinations of material and procedures selected for that purpose. Numerous variations of such details can be implied and will be appreciated by those skilled in the art.

### EXAMPLES

### Example 1

Amplification of an Allene Oxide Cyclase Gene from C. sativa (aoccs)

C. sativa Mill. Cv Vimeiro (resistant ink disease variety) leaves at different infection stages with a virulent race of P. cinnamomi were frozen in liquid nitrogen, grounded to a fine powder in a mortar and stored at −80° C. About 3 g of powder were mix with 20 mL of RNA extraction buffer for RNA extraction, according to the hot borate protocol (Wan and Wilkins, 1994, Anal. Biochem., 223: 7-12). Messenger RNA (mRNA) isolation was preformed with the Poly A Ttract System (Promega) according to manufacturer instructions. The RNA and mRNA pellet was stored in DEPC treated water at −80° C. Spectrophotometric quantification was performed in TE buffer. RNA and mRNA were electrophoresed on a 0.8% agarose gel at 80 V for 1.5 hours to check its integrity.

For the reverse transcription reaction (RT), 1 μg of mRNA and 1 U of Avian Myeloblastosis Virus (AMV) reverse transcriptase in a reaction mixture of 50 mM Tris-HCl pH 8.5, 8 mM MgCl₂, 30 mM KCl and 1 mM DTT, containing 1.0 mM of each dNTP, 12.5 μg actinomicin D and 10 μM of oligo (dT) 17 (provided with 5′/3′ RACE kit, Roche) were incubated for 90 minutes at 55° C. The cDNA produced was amplified with 2.0 U of Taq DNA polymerase (Invitrogen) in a 20 mM Tris-HCl pH 8.4 and 50 mM KCl mixture containing 2.0 mM MgCl₂, 0.25 mM of each dNTP and 10 pmol of each degenerated primers AOCfwd and AOCrev (Table 1). After an initial 5 minutes denaturation period at 94° C., the PCR parameters were 30 seconds template denaturation at 94° C., 45 seconds primer annealing at 45° C. and 2 minute primer extension at 72° C. for 35 cycles. A final extension step of 10 minutes at 72° C. was used subsequently to ensure full-length amplification products. The termocycler used was a Perkin Elmer-Gene Amp PCR System 2400.

The obtained product were purified from the agarose gel and ligated into the vector pBluescript (KS+) (Stratagene). The ligated mixture was used to transform E. coli DH5α. Transformants were selected on LB agar plates containing ampicilin (100 μL/mL) X-Gal (80 μg/mL) and IPTG (0.5 mM). Plasmid DNA was isolated using alkaline lysis method.

DNA sequencing was performed in an automated sequencer ABI 310 Applied Biosystems, using Big Dye Terminator Cycle Sequencing Ready Reaction kit (Applied Biosystems).

The band obtained by PCR has approximately 450 Pb. The nucleotide sequence was sent to NCBI data bank that was shown to have significant homology with Allene Oxide Cyclase genes isolated from other species. As the obtained sequence corresponds to about 60% of the gene coding region, RACE (Rapid Amplification of cDNA Ends) reaction was performed.

In order to perform 5′ RACE reaction, Marathon kit (Clontech) cDNA synthesis reaction was done using 4 μg of mRNA. The adapter reaction allows the use of AP1 (Adaptor Primer, provided with Marathon kit, Clontech) primer in amplification reaction. Marathon cDNA was amplified with 2.0 U Taq DNA polymerase (Invitrogen) in a 20 mM Tris-HCl pH 8.4 and 50 mM KCl mixture containing 2.0 mM MgCl₂, 0.25 mM of each dNTP and 10 pmol of primers AP1 and AOCrev. After an initial 5 minutes denaturation period at 94° C., the PCR parameters were 30 seconds at 94° C., 45 seconds at 60° C. and 45 seconds at 72° C. for 35 cycles, and a final extension step of 10 minutes at 72° C. The 783 pb PCR product was cloned and sequenced as described above.

To perform 3′ RACE reaction, Marathon cDNA was amplified with 2.0 U Taq DNA polymerase (Invitrogen) in a 20 mM Tris-HCl pH 8.4 and 50 mM KCl mixture containing 2.0 mM MgCl₂, 0.25 mM of each dNTP and 10 pmol of primers AOCfwd and AP1. After an initial 5 minutes denaturation period at 94° C., the PCR parameters were 30 seconds at 94° C., 45 seconds at 60° C. and 45 seconds at 72° C. for 35 cycles, and a final extension step of 10 minutes at 72° C. The 873 pb PCR product was cloned and sequenced as described above.

The AOC nucleotide sequence was sent to NCBI data bank and showed significant homology with AOC genes isolated from other species. The highest homology found at the DNA level using the BLASTn program was 99.8% with tomato mRNA clone # AJ272026.

### Example 2

Amplification of a Cystatin Gene from C. sativa (cystcs)

C. sativa leaf extraction, RNA extraction, mRNA isolation and RT reaction were performed exactly as described for aoccs isolation in example 1.

The cDNA produced was amplified with 2.0 U of Taq DNA polymerase (Invitrogen) in a 20 mM Tris-HCl pH 8.4 and 50 mM KCl mixture containing 2.0 mM MgCl₂, 0.25 mM of each dNTP and 10 pmol of each specific primers Cystfwd and Cystrev (Table 1). After an initial 5 minutes denaturation period at 94° C., the PCR parameters were 30 seconds template denaturation at 94° C., 45 seconds primer annealing at 55° C. and 2 minute primer extension at 72° C. for 35 cycles. A final extension step of 10 minutes at 72° C. was used subsequently to ensure full-length amplification products. The termocycler used was a Perkin Elmer-Gene Amp PCR System 2400.

The obtained product were purified from the agarose gel and ligated into the vector pBluescript (KS+) (Stratagene). The ligated mixture was used to transform E. coli DH5α. Transformants were selected on LB agar plates containing ampicilin (100 μL/mL) X-Gal (80 μg/mL) and IPTG (0.5 mM). Plasmid DNA was isolated using alkaline lysis method.

DNA sequencing was performed in an automated sequencer ABI 310 Applied Biosystems, using Big Dye Terminator Cycle Sequencing Ready Reaction kit (Applied Biosystems).

The band obtained by PCR has approximately 450 pb. The nucleotide sequence was sent to NCBI data bank that was shown to have almost 100% homology with Cystatin mRNA isolated from chestnut (# AJ224331) and contained the coding region. Searches in all the available protein and DNA data banks failed to find 100% homology with any existing clone.

### Example 3

Amplification of a β-1,3-Glucanase Gene from C. sativa (gluccs)

C. sativa leaf extraction, RNA extraction, mRNA isolation and RT reaction were performed exactly as described for aoccs isolation in example 1.

The cDNA produced was amplified with 2.0 U of Taq DNA polymerase (Invitrogen) in a 20 mM Tris-HCl pH 8.4 and 50 mM KCl mixture containing 2.0 mM MgCl₂, 0.25 mM of each dNTP and 10 pmol of each degenerated primers Glucfwd and Glucrev (Table 1). After an initial 5 minutes denaturation period at 94° C., the PCR parameters were 30 seconds template denaturation at 94° C., 45 seconds primer annealing at 45° C. and 2 minute primer extension at 72° C. for 35 cycles. A final extension step of 10 minutes at 72° C. was used subsequently to ensure full-length amplification products. The termocycler used was a Perkin Elmer-Gene Amp PCR System 2400.

The obtained product were purified from the agarose gel and ligated into the vector pBluescript (SK+) (Stratagene). The ligated mixture was used to transform E. coli DH5α. Transformants were selected on LB agar plates containing ampicilin (100 μL/mL) X-Gal (80 μg/mL) and IPTG (0.5 mM). Plasmid DNA was isolated using alkaline lysis method.

DNA sequencing was performed in an automated sequencer ABI 310 Applied Biosystems, using Big Dye Terminator Cycle Sequencing Ready Reaction kit (Applied Biosystems).

The band obtained by PCR has approximately 496 pb. The nucleotide sequence was sent to NCBI data bank that was shown to have significant homology with β-1,3-glucanase genes isolated from other species. As the obtained sequence corresponds to about 48% of the gene coding region, and in order to isolate the whole ORF, new specific primers for were designed, Glu5′rev and Glu3′fwd (Table 1), to perform 5′ RACE (Rapid Amplification of cDNA Ends) and 3′RACE reactions, respectively.

For 5′ RACE reaction, Marathon cDNA was amplified with 2.0 U of Taq DNA polymerase (Invitrogen) in a 20 mM Tris-HCl pH 8.4 and 50 mM KCl mixture containing 2.0 mM MgCl₂, 0.25 mM of each dNTP and 10 pmol of each primers Glu5′rev and AP1. After an initial 5 minutes denaturation period at 94° C., the PCR parameters were 30 seconds template denaturation at 94° C., 45 seconds primer annealing at 55° C. and 2 minute primer extension at 72° C. for 35 cycles and a final extension step of 10 minutes at 72° C. The approximately 600 pb product was cloned and sequenced as described above.

For 3′ RACE reaction, cDNA from an RT performed as described in Example 1 was amplified with 2.0 U of Taq DNA polymerase (Invitrogen) in a 20 mM Tris-HCl pH 8.4 and 50 mM KCl mixture containing 2.0 mM MgCl₂, 0.25 mM of each dNTP and 10 pmol of each primers Glu3′fwd and Vial9 (provided with 5′/3′ RACE kit, Roche). After an initial 5 minutes denaturation period at 94° C., the PCR parameters were 30 seconds template denaturation at 94° C., 45 seconds primer annealing at 55° C. and 2 minute primer extension at 72° C. for 35 cycles and a final extension step of 10 minutes at 72° C. The approximately 300 pb product was cloned and sequenced as described above.

Fused together by PCR amplification, the 613 pb, the 496 pb and the 300 pb sequences represented the complete coding region for C. sativa β-1,3-Glucanase protein. All the three isolated β-1,3-Glucanase fragments together comprise a cDNA molecule of 1374 pb in size and enclose 100% of the coding region. The complete nucleotide sequence was sent to NCBI data bank and showed significant homology with β-1,3-Glucanase genes isolated from other species. The highest homology found at the mRNA level using the BLASTn program was 81% with Vitis vinifera mRNA clone # AF239617. Searches in all the available protein and DNA data banks failed to find 100% homology with any existing clone.

### Example 4

Amplification of a Thaumatin-Like Protein Gene from C. sativa (tlpcs)

C. sativa leaf extraction, RNA extraction, mRNA isolation and RT reaction were performed exactly as described for aoccs isolation in example 1.

The cDNA produced was amplified with 2.0 U of Taq DNA polymerase (Invitrogen) in a 20 mM Tris-HCl pH 8.4 and 50 mM KCl mixture containing 2.0 mM MgCl₂, 0.25 mM of each dNTP and 10 pmol of each specific primers Thaufwd and Thaurev (Table 1). After an initial 5 minutes denaturation period at 94° C., the PCR parameters were 30 seconds template denaturation at 94° C., 45 seconds primer annealing at 55° C. and 2 minute primer extension at 72° C. for 35 cycles. A final extension step of 10 minutes at 72° C. was used subsequently to ensure full-length amplification products. The termocycler used was a Perkin Elmer-Gene Amp PCR System 2400.

The obtained product were purified from the agarose gel and ligated into the vector pBluescript (SK+) (Stratagene). The ligated mixture was used to transform E. coli DH5α. Transformants were selected on LB agar plates containing ampicilin (100 μL/mL) X-Gal (80 μg/mL) and IPTG (0.5 mM). Plasmid DNA was isolated using alkaline lysis method.

DNA sequencing was performed in an automated sequencer ABI 310 Applied Biosystems, using Big Dye Terminator Cycle Sequencing Ready Reaction kit (Applied Biosystems).

The band obtained by PCR has approximately 550 pb. The nucleotide sequence was sent to NCBI data bank that was shown to have significant homology with Thaumatin-Like Protein genes to have almost 100% homology with Thaumatin-Like Protein mRNA isolated from chestnut (# AJ242828) and contained 77.1% of the coding region. In order to isolate the whole ORF, new specific primers for were designed, Thau5′fwd and Thau3′rev (Table 1), to perform 5′ RACE and 3′RACE reactions, respectively.

For 5′ RACE reaction, Marathon cDNA was amplified with 2.0 U of Taq DNA polymerase Advantage 2 (Clontech) in a 20 mM Tris-HCl pH 8.4 and 50 mM KCl mixture containing 2.0 mM MgCl₂, 0.25 mM of each dNTP and 10 pmol of each primers Thau5′fwd and Thaurev. After an initial 2 minutes denaturation period at 94° C., the PCR parameters were 30 seconds template denaturation at 94° C., 45 seconds primer annealing at 55° C. and 2 minute primer extension at 72° C. for 35 cycles and a final extension step of 10 minutes at 72° C. The approximately 750 pb product was cloned and sequenced as described above.

For 3′ RACE reaction, Marathon cDNA was amplified with 2.0 U of Taq DNA polymerase Advantage 2 (Clontech) in a 20 mM Tris-HCl pH 8.4 and 50 mM KCl mixture containing 2.0 mM MgCl₂, 0.25 mM of each dNTP and 10 pmol of each primers Thaufwd and Thau3′rev. After an initial 2 minutes denaturation period at 94° C., the PCR parameters were 30 seconds template denaturation at 94° C., 45 seconds primer annealing at 55° C. and 2 minute primer extension at 72° C. for 35 cycles and a final extension step of 10 minutes at 72° C. The approximately 600 pb product was cloned and sequenced as described above.

Fused together by ligation, the 750 pb and the 600 pb sequences represented the complete coding region for C. saliva Thaumatin-Like Protein. The two isolated Thaumatin-Like Protein fragments together comprise a cDNA molecule of 806 pb in size and enclose 100% of the coding region. The complete nucleotide sequence was sent to NCBI data bank and showed significant homology with Thaumatin-Like Protein genes isolated from other species, and was shown to have almost 100% homology with Thaumatin-Like Protein mRNA isolated from chestnut (#AJ242828) and contained the coding region. Searches in all the available protein and DNA data banks failed to find 100% homology with any existing clone.

The specific primers for 5′ and 3′ RACE were designed using as template the nucleic acid sequences previously obtained by PCR, in each of the examples. Table 1 presents all the designed primers used for gene isolation.

TABLE 1

Primer sequence designed for this invention.

Primer

| | |
|---|---|
| designation | Primer sequence (5′→3′) |

| | |
|---|---|
| AOCfwd | CG(A/T/C)GA(C/T)(A/C)G(A/T/C)G(A/G) |
| | (G/A/T)AG(C/T)CC(A/T/C)GC(A/T/C)TA |
| AOCrev | GC(C/T)TT(A/G)GC(G/A/T)(G/T)C(G/A/T)G |
| | (G/T)(A/T/C)G(A/T/C)TGG(C/T)TC |
| Cystfwd | GAGAAAAATGGCAGCACTAGTTGGAGGAG |
| Cystrev | GAGAAAAATGGCAGCACTAGTTGGAGGAG |
| Glucfwd | A(A/G)A(C/T)(A/C)A(A/G)ATCAA(A/G)GT |
| | (C/T)TC(C/T)ACTGC |
| Glucrev | AAACA(A/T)(A/G)GC(A/G)AA(A/T)A(A/T) |
| | (A/G)TAAG(C/T)CTC |
| Gluc5′rev | CTCTGAAAGCCCCTGCTGACGGA |
| Gluc3′fwd | CAGCAGGTGGATTCGCTACATCA |
| Thaufwd | AACCTCAACTACCGAACACTGGAT |
| Thaurev | AGGTAAAGGTGCTGGTAGAATCA |
| Thau5′fwd | CCAAACCCAAGTTCATCG |
| Thau3′rev | GGGAACGCATAATTCCTCTC |

1. Four isolated nucleic acid sequences from Castanea sativa Mill. Comprising encoding regions for Allene Oxide Cyclase (AOCCs), Cystatin (CystCs), β-1,3-Glucanase (GlucCs) and Thaumatin-Like Protein (TLPCS) proteins.

2. The isolated nucleic acid molecule, according to claim 1, wherein the polynucleotide has the sequence of SEQ. ID. NO: 1.

3. The isolated nucleic acid molecule, according to claim 2, wherein the polynucleotide encodes an Allene Oxide Cyclase polypeptide.

4. The isolated nucleic acid sequences according to claim 2, wherein the polynucleotide encodes a protein or polypeptide having an amino acid sequence of SEQ. ID. NO: 2.

5. The isolated nucleic acid molecule, according to claim 1, wherein the polynucleotide has the sequence of SEQ. ID. NO: 3.

6. The isolated nucleic acid molecule, according to claim 5, wherein the polynucleotide encodes a Cystatin polypeptide.

7. The isolated nucleic acid sequences according to claim 5, wherein the polynucleotide encodes a protein or polypeptide having an amino acid sequence of SEQ. ID. NO: 4.

8. The isolated nucleic acid molecule, according to claim 1, wherein the polynucleotide has the sequence of SEQ. ID. NO: 5.

9. The isolated nucleic acid molecule, according to claim 8, wherein the polynucleotide encodes a β-1,3-Glucanase polypeptide.

10. The isolated nucleic acid sequences according to claim 8, wherein the polynucleotide encodes a protein or polypeptide having an amino acid sequence of SEQ. ID. NO: 6.

11. The isolated nucleic acid molecule, according to claim 1, wherein the polynucleotide has the sequence of SEQ. ID. NO: 7.

12. The isolated nucleic acid molecule, according to claim 11, wherein the polynucleotide encodes a Thaumatin-Like Protein polypeptide.

13. The isolated nucleic acid sequences according to claim 11, wherein the polynucleotide encodes a protein or polypeptide having an amino acid sequence of SEQ. ID. NO: 8.

14. The isolated nucleic acid sequences according to claim 1, presented as RNA, mRNA, cRNA, DNA or cDNA molecules.

15. The isolated nucleic acid sequences described in claim 1, which can be used together with other genes expressed in Castanea sativa Mill.

16. A chimeric gene comprising one or more nucleic acid molecules according to claim 1 in sense orientation and which can be operably linked to a promoter.

17. Any expression cassette comprising one of the chimerical genes described in claim 16.

18. Any replicable expression vector comprising one of the chimerical genes described in claim 16.

19. A plant genome comprising one of the chimerical genes described in claim 16.

20. A host cell transformed with one of the chimerical genes described in claim 16.

21. A genetically modified plant containing one of the chimerical genes described in claim 16, wherein said chimerical gene is stably integrated into the plant genome.

22. The progeny of cross breeding involving the plant described in claim 21.

23. The fruit or seeds comprising one of the chimerical genes described in claim 16, wherein said chimerical gene, is stably integrated into the plant genome.

24. Any method of improving the defence response signalling to the ink disease, the method comprising introduction into the plant of an expression cassette according to the described in claim 17.

25. Any method of improving the counteract of fungal protease action, the method comprising introduction into the plant of an expression cassette according to the described in claim 17.

26. Any method of improving the attack of the fungal cell wall, the method comprising introduction into the plant of an expression cassette according to the described in claim 17.

27. Any method of improving the permeability and rupture of the fungal cellular membrane, the method comprising introduction into the plant of an expression cassette according to the described in claim 17.


SEQUENCE LISTING (8 sequences)

SEQ ID NO: 1 - DNA, 735 bp, Castanea sativa
atggccactgtttcctcagcctctgctgctcttagaaccatttcttcttcctcatccaagctatcttctgccttccaaactaaaaagatccaatcttttaaactacctaaccctctcatttctcagaatcataaacttactaccacctctactactgcttccagatcattttcctgcaagagccagagcacctcaacagattcaactaacactgaagttcaagaacttagtgtctatgagatcaatgaacgtgatcgtggaagccctgcttatcttcgattgagccaaaagactgttaattcactcggagatcttgtcccctttagcaacaagctatatactgcagatctaaagaagagaattggaataacagcaggactctgcattctgatcaagcacgaagaagagaagaaaggagatcgctatgaagctgtttacagcttctacttcggcgattacggtcatatcgccgttcagggagcgtacttaacctatgaagaaacttacctagccgtcaccggtggatccggcatatttgcaggggtttccggtcaagtgaaattgcagcaactcattttccctttcaagctattttacactttttacttgaaggggatccccggtctgccatctgaattgttgtgtacggcggttcctccgtcgccgacggtggagccaacacctgaagctaaagcttgtgaggaaggggccgcactgaaaaattacactaattaa

SEQ ID NO: 2 - protein, 244 aa, Castanea sativa
MATVSSASAALRTISSSSSKLSSAFQTKKIQSFKLPNPLISQNHKLTTTSTTASRSFSCKSQSTSTDSTNTEVQELSVYEINERDRGSPAYLRLSQKTVNSLGDLVPFSNKLYTADLKKRIGITAGLCILIKHEEEKKGDRYEAVYSFYFGDYGHIAVQGAYLTYEETYLAVTGGSGIFAGVSGQVKLQQLIFPFKLFYTFYLKGIPGLPSELLCTAVPPSPTVEPTPEAKACEEGAALKNYTN

SEQ ID NO: 3 - DNA, 318 bp, Castanea sativa
atgagaaaattggcagcactagttggaggagtgtcagatgttaagggacatgagaacagcttgcagatcgacgacctcgctcgttttgctgtcgacgaccacaacaagaaagcgaatacactgctgcagtttaagaaggtggtgaatgcgaaacagcaggtggtttctggaacaatatacattctaacgttggaggtggaggatggcgggaagaagaaagtttatgaagccaagatttgggagaagccatggttgaacttcaaggaggtgcaggaatttaagctcattggtgatgcccctacacaccatagtgcttaa

SEQ ID NO: 4 - protein, 105 aa, Castanea sativa
MRKLAALVGGVSDVKGHENSLQIDDLARFAVDDHNKKANTLLQFKKVVNAKQQVVSGTIYILTLEVEDGGKKKVYEAKIWEKPWLNFKEVQEFKLIGDAPTHHSA

SEQ ID NO: 5 - DNA, 927 bp, Castanea sativa
atgagaatctatgatccgaatcaggctgttctacaagcccttagaggctctaacattgaagtcatgctaggtgtcccaaactcagaccttcaaagccttgccaacccttccaatgcacaagcatgggtacaaagaaacgtacttaacttctggcctagtgtcaggtttcgatacattgcagttggaaatgaagtgagtcctgttaatggaggcacagctgggttagcacaaattgttctccctgccttaaccaacgtattcaatgcaattagatcagctggccttaaggaccaaatccaggtttcaattgcaattgacatgaccttaataggaaactcctatcctccgtcagcaggggctttcagaggtgatgttagatcatatttagacccaatcattggtcacctagtatatgctaaggcacccttactagccaatgtgtacacttattttagctatgctggaaatccacgcgacatttctcttccctatgctttgtttacttccccatcagttgtggcatgggatggtcctaagggataccaaaacctttttgatgcaatgatggatgcattgtactcagctctcgagaggtcgtggggcggttcattggaggttgttgtttcagagagtggatggccatcagcagcaggtggattcgctacatcatttgataatgcacgtacttattactcaaatttgattaagcatgtcaaagggggtacaccaaagaggcctgggggagctatagagacctatctttttgccatgtttaatgagaatcagaaacaaccagagctagagaaaaactttggcttattcttcccaaataaacagcccaaatttaacctcaattttggtggagaaagaatctgggatgtcactgctgaatataatgcaacagtttccctcagtagtgatatgtaa

SEQ ID NO: 6 - protein, 308 aa, Castanea sativa
MRIYDPNQAVLQALRGSNIEVMLGVPNSDLQSLANPSNAQAWVQRNVLNFWPSVRFRYIAVGNEVSPVNGGTAGLAQIVLPALTNVFNAIRSAGLKDQIQVSIAIDMTLIGNSYPPSAGAFRGDVRSYLDPIIGHLVYAKAPLLANVYTYFSYAGNPRDISLPYALFTSPSVVAWDGPKGYQNLFDAMMDALYSALERSWGGSLEVVVSESGWPSAAGGFATSFDNARTYYSNLIKHVKGGTPKRPGGAIETYLFAMFNENQKQPELEKNFGLFFPNKQPKFNLNFGGERIWDVTAEYNATVSLSSDM

SEQ ID NO: 7 - DNA, 732 bp, Castanea sativa
atgaaaaccctggcactctacggccttaccttggccttctttttcttatctggtgcacactctgctaaaataactttcacaaacaactgtccaagaaccatctggccaggaaccctaacttcggatcaaaaacctcaactttcaaaaactggatttgagttagcatccaaagcatccttaacattggaatgttcaagctccatggaaaggccggttttgggcccgaacccgatgcaccaccaaatcaggaaagttcacttgcgagactgctgattgtagcaccggtcaggttgcatgcaatggtaacggtgcaatcccaccagcttctttagtagaaatcaacatagcagccaatcgtgggatggactattatgatgttagccttgtagatggcttcaacttgcctgtttctgtagccaccagaggcggcactggtgattgcaaggccacaagctgtccagctaatgtgaacgcagtttgcccagcggaattacaagtgaaagggtctgatgggagcgtgcttgcttgcaagagcgcttgtattgctttcaatcaaccacagtactgttgcactggtgcatttaacaccccgaaaacatgtccacccacaaaatattctcgcatctttaagcaacaatgtcctcaagcttatagctatgcttatgatgatcctaccagcacctttacctgctcaagtgcacccgactatgttatcgcattttgtccataaat

SEQ ID NO: 8 - protein, 226 aa, Castanea sativa
MKTLALYGLTLAFFFLSGAHSAKITFTNNCPRTIWPGTLTSDQKPQLSKTGFELASKASLTLECSSSMERPVLGPNPMHHQIRKVHLRDCHRSGCMQWRCNPTSFFSRNQHSSQSWDGLLPCRWLQLACFCSHQRRHWLQGHKLSSCERSLPSGITSERVWERACLQERLYCFQSTTVLLHWCIHPENMSTHKIFSHLATMSSSLSYQHLYLLKCTRLCYRILSIN